(19) United States
(12) Patent Application Publication
(10) Pub. No.: US 2022/0326210 A1
(43) Pub. Date: Oct. 13, 2022

(54) ENHANCED CHEMICAL AND BIOLOGICAL DETECTIONS WITH SIZE-SHRINKABLE HYDROGELS

(71) Applicants: Ming Su, Newton, MA (US); Yiting Zheng, Boston, MA (US)

(72) Inventors: Ming Su, Newton, MA (US); Yiting Zheng, Boston, MA (US)

(21) Appl. No.: 17/717,994

(22) Filed: Apr. 11, 2022

Related U.S. Application Data
(60) Provisional application No. 63/173,002, filed on Apr. 9, 2021.

(51) Int. Cl. G01N 33/18; G01N 31/22; G01N 21/64

(52) U.S. Cl. CPC G01N 33/1813; G01N 31/22; G01N 21/643; G01N 2021/6439

(57) ABSTRACT

Disclosed is a method for detecting a metal ion in an aqueous solution, the method comprising immersing a hydrogel in the aqueous solution, wherein the hydrogel comprises a prefluorescent imaging moiety that is activated upon binding the metal ion. The method enables detection of lead ions in water.

### RELATED APPLICATION

This application claims the benefit of priority to U.S. Provisional Patent Application No. 63/173,002, filed Apr. 9, 2021.

### BACKGROUND

Lead ion is a devastating environmental toxin (Wani, A. L. et al. 2015; Sanders, T. et al. 2009). Analytical methods to detect aqueous lead ions include atomic absorption spectrometry, inductively coupled plasma mass spectrometry, and optical emission spectrometry (Madrid, Y. et al. 1994; Chen, J. et al. 2001; Rapsomanikis, S. et al. 1986; Greda, K. et al. 2016), but these methods are expensive and require trained personnel to operate them. Fluorescent methods are often used to detect lead ions in aqueous and organic solutions due to their operational readiness (Deo, S. et al. 2000; Wan, J. et al. 2017; Dhenadhayalan, N. et al. 2016; Kim, Y. et al. 2015; Li, Z.-h. et al. 2017). However, existing fluorescent probes can optically detect lead ions at a sufficiently high concentration (>10⁻⁵ M) because of their low sensitivity. In the case of monolayer-based detection, an even higher concentration of fluorescent probes (>10⁻⁴ M) is needed (Ju, H. et al. 2011), which is much higher than the national standard for lead levels in drinking water.

The low sensitivity in fluorescent detection is because of low concentration of metal ions in a given volume of optical excitation. Preconcentration such as extraction can be used to enrich low-concentration metal ions, but it needs more sample and extra operation steps and may lead to contamination (Wu, J. et al. 1997; Mandil, A. et al. 2010; Shi, D. et al. 2016; Goryacheva, I. Y. et al. 2005; Javed, H. et al. 2018). If the probes can be enriched after binding to metal ions, the sensitivity can be enhanced without altering optical illumination and fluorescent detectors (Chen, F. et al. 2015). One way of increasing local concentration of fluorescent probes is to take advantage of size-changeable hydrogels, whose volume can be reduced hundreds or even thousands of times once dehydrated (Wu, Z. L. et al. 2013; Gong, J. et al. 2020).

The idea of volume increase of hydrogels has not been used to enhance metal ion detection when the volume of hydrogel decreases. Agarose gel shrinks more significantly after dehydration than other hydrogels such as polyacrylamide gel and gelatin (Garner, B. W. et al. 2009; Burnham, M. R. et al. 2006; Futscher, M. H. et al. 2017), while agarose lacks active moieties (i.e., amine, carboxyl, or thiol groups) and cannot be modified directly (Ingavle, G. C. et al. 2014). Agarose can be activated to form carboxylated agarose (CA) by oxidizing its primary alcohol groups, and CA can form a stable hydrogel at room temperature (Su, Y. et al. 2013; Forget, A. et al. 2015; Hu, J.-X. et al, 2016; Pierre, G. et al. 2017).

Hydrogels with their biocompatibility and rich surface chemistry are also promising candidates as biosensing platforms (Applewood, D. C. et al. 2011; Savina, I. N. et al. 2016; Meiring, J. E. et al. 2004; Zubtsov, D. A. et al. 2007). The 3D constructs of hydrogels allow immobilization of capturing probes and target analytes with high binding capacity over flat surfaces and thus allow detection of analytes at even lower concentration. However, a major issue associated with hydrogel is that the interlaced networks with randomly oriented pores in the hydrogel made by electrospinning, gas foaming, freeze-thaw and salt templating [Burdick, J. A. et al. 2011; Dadsetan, M. et al. 2008; Dehghani, F. et al. 2011; De France, K. J. et al. 2018; Kumar, A. et al. 2010; Madihally, S. V. et al. 1999) decrease the diffusivity and permeability of molecules, especially biomolecules and large reporters such as nanoparticles (Sandrin, D. et al. 2016; Azpe, E. et al. 2019).

Nanoparticles with unique physical properties have been used as highly sensitive probes to detect biomarkers—antigens, proteins, and nucleic acids—by binding the specific biomarkers to the capturing agents immobilized on nanoparticle surfaces, which alters the physical properties of the nanoparticles. Taking optical property as an example, upon excitation by high energy photons, nanoparticles emit low energy photons as fluorescence emission which is detected after filtering excitation photons. While the sensitivity of nanoparticle-based biomarker detection is relatively high compared to that of organic fluorophores, nanoparticles immobilized on the surface form a monolayer and the number of nanoparticles contributing to fluorescence emission is still low (Lee, H. J. et al. 2008; Lenzi, E. et al. 2019).

### SUMMARY OF INVENTION

The present invention provides a method for detecting a metal ion in an aqueous solution, the method comprising:
(1) immersing a hydrogel in the aqueous solution,
   wherein the hydrogel comprises a latent imaging moiety that is activated upon binding the metal ion;
(2) removing the hydrogel from the aqueous solution;
(3) dehydrating the hydrogel; and
(4) detecting the activated imaging moiety.

The present invention also provides a method for detecting a metal ion in an aqueous solution, the method comprising:
(1) immersing a hydrogel in the aqueous solution,
   wherein the hydrogel comprises a latent prefluorescent imaging moiety that is activated upon binding the metal ion;
(2) removing the hydrogel from the aqueous solution;
(3) dehydrating the hydrogel; and
(4) detecting the fluorescence emitted by the activated prefluorescent imaging moiety.

The present invention further provides a method for detecting a metal ion in water, the method comprising:
(1) immersing a hydrogel in the water,
   wherein the hydrogel comprises a latent imaging moiety that activated upon binding the metal ion;
(2) removing the hydrogel from the water;
(3) dehydrating the hydrogel; and
(4) detecting the activated imaging moiety.

The present invention further provides a method for detecting a metal ion in water, the method comprising:
(1) immersing a hydrogel in the water,
   wherein the hydrogel comprises a latent prefluorescent imaging moiety that is activated upon binding the metal ion;
(2) removing the hydrogel from the water;
(3) dehydrating the hydrogel; and
(4) detecting the fluorescence emitted by the activated prefluorescent imaging moiety.

The present invention further provides a method for detecting a biomarker in an aqueous solution, the method comprising:
(1) immersing a hydrogel in the aqueous solution,
   wherein the hydrogel comprises a first biomarker capturing agent moiety;
(2) adding an imaging probe to the aqueous solution,
   wherein the imaging probe comprises a second biomarker capturing agent moiety;
(3) removing the hydrogel from the aqueous solution;
(4) dehdyrating the hydrogel; and
(5) detecting the imaging probe.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1: Scheme of enhanced turn-on fluorescence detection of lead ions with a dehydrating hydrogel.

FIG. 2A: Structures of agarose and modified agaroses containing modified rhodamine moiety.

FIG. 2B: FTIR spectra of agarose (lower), carboxylated agarose (middle), and fluorescent probe-immobilized agarose (B upper).

FIG. 3A: Digital images of metal ion solutions under different conditions: with the prefluorescence probe under natural light (upper), without the prefluorescence probe but under UV excitation (middle), and with the prefluorescence probe and under UV excitation (lower).

FIG. 3B: Fluorescent spectra with different metal ion including iron, copper, calcium, cobalt, and lead.

FIG. 3C: Time-dependent intensity of 10⁻⁴ M metal ion solutions containing the prefluorescence probe.

FIG. 3D: pH-dependent intensity of 10⁻⁴ M metal ion solutions containing the prefluorescence probe.

FIG. 4A: Dehydrated volume of a 15 mL hydrogel with different amounts of agarose.

FIG. 4B: Optical images of the hydrogel before (left) and after (right) dehydration under UV light.

FIG. 4C: Fluorescence micrographs of the dehydrated hydrogel with physically absorbed rhodamine 6G (left) and with immobilized fluorescent probe (right).

FIG. 4D: The relationship between fluorescent intensity and lead ion concentrations.

FIG. 5A: Fluorescence micrographs of the hydrogel (A upper), after dehydration (A middle), and selected regions for intensity analysis (A lower) (exposure time: 100 ms);

FIG. 5B: Fluorescent intensity as a function of lead ion concentration before (black, upper left) and after (red, lower right) dehydration.

FIG. 5C: Fluorescent intensity as a function of hydrogel thickness at different lead ion concentrations with vertical dehydration.

FIG. 6A: Generation of hydrogel with uniaxial channel scaffold. Auniaxial temperature gradient was created by exposing the bottom surface of the hydrogel column to a copper sheet on liquid nitrogen.

FIG. 6B: Fluorescent images of transverse (left) and longitudinal (right) cross-sections of scaffold.

FIG. 6C: SEM images of transverse and longitudinal cross sections of scaffold after lyophilization.

FIG. 7A: Time-lapse diffusion distance of molecules in hydrogels and scaffolds.

FIG. 7B: Optical images of CA-NA scaffold before dehydration and after dehydration

FIG. 7C: Fluorescent images of transverse cross section of scaffold during dehydration.

FIG. 7D: The relationship between water weight ratio in the matrix and time during dehydration.

FIG. 8A: ATR-FTIR spectra of surface modified iron oxide nanoparticles.

FIG. 8B: The relationship between primary x-ray beam diameter and distance.

FIG. 8C: X-ray fluorescence spectra of iron oxide nanoparticle samples

FIG. 9A: Count of secondary X-ray fluorescence before and after dehydration for the scaffold samples with different thickness.

FIG. 9B: The relationship between the count of secondary X-ray fluorescence and avidin concentration

FIG. 10: Enhanced X-ray fluorescence detection of biomarkers with dehydrating hydrogel.

### DETAILED DESCRIPTION OF THE INVENTION

This present invention discloses a signal enhancement mechanism based on size-shrinkable matrix, i.e., size-shrinkable hydrogel, for chemical and biological sensing, in which the signal intensity is amplified by shrinking the size of matrix so that signal can be enhanced by several times. This method can be used for chemical detection and biological detection.

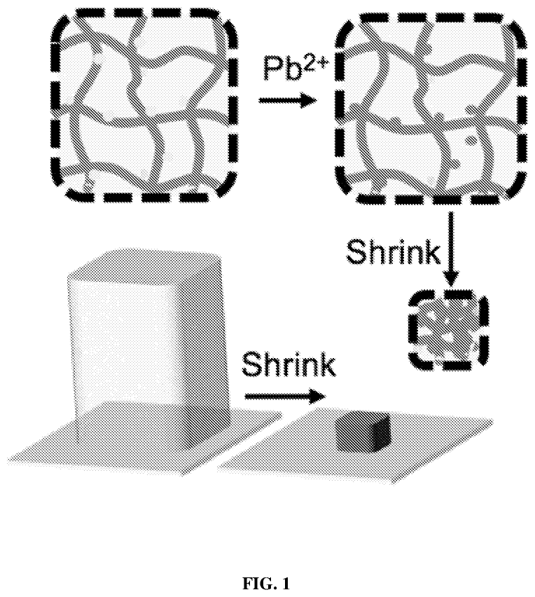

The present inventions provides a method to detect aqueous lead ions at low concentrations with turn-on fluorescence on a size-shrinkable hydrogel (FIG. 1). A prefluorescent molecule was immobilized on the hydrogel (Guenther, M. et al. 2007; Xue, F. et al. 2014). Once selective binding of the prefluorescent molecule to lead ions occurs, the fluorescent signal turned on. By then shrinking the volume of the hydrogel where the fluorescence probes are chemically linked, the sensitivity of detection was enhanced, indicating that the fluorescent quench at a high fluorophore concentration does not play a significant role.

The prefluorescent probe used herein is a derivative of rhodamine, which is grafted onto the carboxylated agarose (CA) backbone through an amine moiety. Once bound to target metal ions, the molecules undergo a structural change from a spirocyclic lactam to an open-ring amide (Czaplyski, W. L. et al. 2014; Yin, J. et al. 2013), resulting in pink fluorescence emission. The agarose hydrogel shrinks upon dehydration, and the density of fluorescent probes increases in the gel, causing fluorescence signals to be enhanced.

Advantages of the disclose method include low cost, high sensitivity, and non-toxicity. The method is also field deployable and easy to operate.

This present invention also provides a method to enhance the sensitivity of nanoparticle-based protein biomarker detection by exploiting volume reduction of hydrogels upon dehydration. Carboxylated agarose hydrogels with uniaxial channels allow for high diffusivity of biomolecules and fast dehydration. Protein biomarkers, collected from a sample solution and captured on the surface of hydrogel channels, have been detected based on characteristic X-ray fluorescence of nanoparticles, which are modified with ligands of the biomarkers to create a one-to-one correspondence.

Once the nanoparticles bind to the biomarkers captured on the in the hydrogel scaffold, dehydration of the hydrogel scaffold leads to a size reduction over 80 times, and an increase of nanoparticle amount in the interaction volume of primary x-ray beam, leading to the enhanced intensity of X-ray fluorescence imaging. By determining the presence and concentration of the nanoparticles using X-ray fluorescence, the amount of biomarkers can be detected with limits of 2 μg/mL.

### EXEMPLARY EMBODIMENTS OF THE INVENTION

The present invention provides a method for detecting a metal ion in an aqueous solution, the method comprising:
(1) immersing a hydrogel in the aqueous solution,
   wherein the hydrogel comprises a latent imaging moiety that is activated upon binding the metal ion;
(2) removing the hydrogel from the aqueous solution;
(3) dehydrating the hydrogel; and
(4) detecting the activated imaging moiety.

In certain embodiments, the method further comprising (5) determining the concentration of the metal ion in the aqueous solution.

In certain embodiments, the metal ion is a Pb²⁺ ion.

In certain embodiments, the hydrogel is size, e.g. volume, shrinkable.

In certain embodiments, the hydrogel shrinks upon dehydration.

The present invention also provides a method for detecting a metal ion in an aqueous solution, the method comprising:
(1) immersing a hydrogel in the aqueous solution,
   wherein the hydrogel comprises a latent prefluorescent imaging moiety that is activated upon binding the metal ion;
(2) removing the hydrogel from the aqueous solution;
(3) dehydrating the hydrogel; and
(4) detecting the fluorescence emitted by the activated prefluorescent imaging moiety.

In certain embodiments, the method further comprising (3a) exposing the dehydrated hydrogel to UV light.

In certain embodiments, the method further comprising (5) determining the concentration of the metal ion in the aqueous solution.

In certain embodiments, the concentration of the metal ion in the aqueous solution and the intensity of the fluorescence of the activated prefluorescent imaging moiety have a linear relationship.

In certain embodiments, the metal ion is a Pb²⁺ ion.

In certain embodiments, the hydrogel is size shrinkable.

In certain embodiments, the hydrogel shrinks upon dehydration.

In certain embodiments, in step (1), the hydrogel is immersed in the aqueous solution for about 20 to about 60 min.

In certain embodiments, the hydrogel is immersed in the aqueous solution for about 30 min.

In certain embodiments, in step (1), the aqueous solution containing the immersed hydrogel is heated to about 50 to 80° C.

In certain embodiments, the aqueous solution containing the immersed hydrogel is heated to about 60° C.

In certain embodiments, the hydrogel expands upon immersion on the aqueous solution.

In certain embodiments, in step (2), the hydrogel is dehydrated at about 60 to about 90° C.

In certain embodiments, the hydrogel is dehydrated at about 70° C.

In certain embodiments, the hydrogel is dehydrated for about 20 to about 60 min.

In certain embodiments, the hydrogel is dehydrated for about 30 min.

In certain embodiments, the volume of the hydrogel is reduced by about 30 to about 50 times upon dehydration.

In certain embodiments, the volume of the hydrogel is reduced by about 40 times upon dehydration.

In certain embodiments, the intensity of the fluorescence emitted by the activated prefluorescent imaging moiety in the dehydrated hydrogel is about 5 to about 15 times greater relative to the wet hydrogel.

In certain embodiments, the intensity of the fluorescence emitted by the activated prefluorescent imaging moiety in the dehydrated hydrogel is about 10 times greater relative to the wet hydrogel.

In certain embodiments, the hydrogel comprises a modified agarose polymer.

In certain embodiments, the hydrogel comprises a carboxylated agarose polymer. In certain embodiments, the molecular weight of the carboxylated agarose polymer is 50,000-200,000, 50,000-150,000, 75,000-150,000, 80,000-140,000, 100,000-150,000, or about 120,000 amu.

In certain embodiments, each monomer of the carboxylated agarose polymer has the following structure:

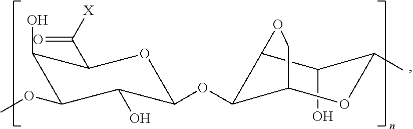

wherein X is the prefluorescent imaging moiety; n is an integer; and the molecular weight of the carboxylated agarose polymer is 50,000-200,000, 50,000-150,000, 75,000-150,000, 80,000-140,000, 100,000-150,000, or about 120,000 amu.

In certain embodiments, the latent prefluorescent imaging moiety comprises a modified rhodamine moiety.

In certain embodiments, the modified rhodamine moiety comprises a tris(2-aminoethyl)amino moiety.

In certain embodiments, the latent prefluorescent imaging moiety has the following structure:

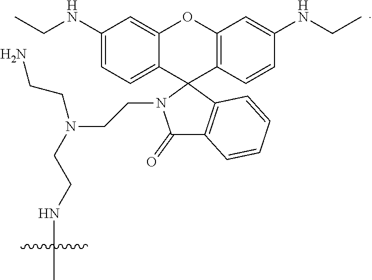 [SMILES: *NCCN(CCN)CCN1C(=O)c2ccccc2C12c1ccc(NCC)cc1Oc1cc(NCC)ccc12]

In certain embodiments, the activated prefluorescent imaging moiety that is activated upon binding to the metal ion, has the following structure:

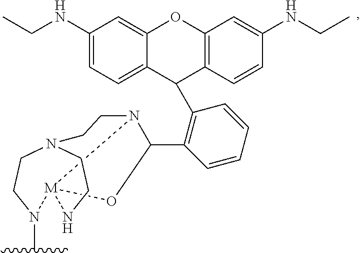

wherein M is a metal ion.

The present invention also provides a method for detecting a metal ion in water, the method comprising:
(1) immersing a hydrogel in the water,
   wherein the hydrogel comprises a latent imaging moiety that activated upon binding the metal ion;
(2) removing the hydrogel from the water;
(3) dehydrating the hydrogel; and
(4) detecting the activated imaging moiety.

In certain embodiments, the method further comprising (5) determining the concentration of the metal ion in the water.

In certain embodiments, the metal ion is a Pb²⁺ ion.

In certain embodiments, the hydrogel is size shrinkable.

In certain embodiments, the hydrogel shrinks upon dehydration.

The present invention further provides a method for detecting a metal ion in water, the method comprising:
(1) immersing a hydrogel in the water,
   wherein the hydrogel comprises a latent prefluorescent imaging moiety that is activated upon binding the metal ion;
(2) removing the hydrogel from the water;
(3) dehydrating the hydrogel; and
(4) detecting the fluorescence emitted by the activated prefluorescent imaging moiety.

In certain embodiments, the method further comprising (3a) exposing the dehydrated hydrogel to UV light.

In certain embodiments, the method comprising (5) determining the concentration of the metal ion in the water.

In certain embodiments, the concentration of the metal ion in the water and the intensity of the fluorescence of the activated prefluorescent imaging moiety have a linear relationship.

In certain embodiments, the metal ion is Pb²⁺ ion.

In certain embodiments, the hydrogel is size shrinkable.

In certain embodiments, the hydrogel shrinks upon dehydration.

In certain embodiments, in step (1), the hydrogel is immersed in the water for about 20 to about 60 min.

In certain embodiments, the hydrogel is immersed in the water for about 30 min.

In certain embodiments, in step (1), the water containing the immersed hydrogel is heated to about 50 to 80° C.

In certain embodiments, the water containing the immersed hydrogel is heated to about 60° C.

In certain embodiments, the hydrogel expands upon immersion on the water.

In certain embodiments, in step (2), the hydrogel is dehydrated at about 60 to about 90° C.

In certain embodiments, the hydrogel is dehydrated at about 70° C.

In certain embodiments, the hydrogel is dehydrated for about 20 to about 60 min.

In certain embodiments, the hydrogel is dehydrated for about 30 min.

In certain embodiments, the volume of the hydrogel is reduced by about 30 to about 50 times upon dehydration.

In certain embodiments, the volume of the hydrogel is reduced by about 40 times upon dehydration.

In certain embodiments, the intensity of the fluorescence emitted by the activated prefluorescent imaging moiety in the dehydrated hydrogel is about 5 to about 15 times greater relative to the wet hydrogel.

In certain embodiments, the intensity of the fluorescence emitted by the activated prefluorescent imaging moiety in the dehydrated hydrogel is about 10 times greater relative to the wet hydrogel.

In certain embodiments, the hydrogel comprises a modified agarose polymer.

In certain embodiments, the hydrogel comprises a carboxylated agarose polymer. In certain embodiments, the molecular weight of the carboxylated agarose polymer is 50,000-200,000, 50,000-150,000, 75,000-150,000, 80,000-140,000, 100,000-150,000, or about 120,000 amu.

In certain embodiments, each monomer of the carboxylated agarose polymer has the following structure:

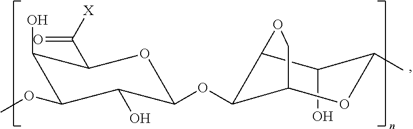

wherein X is the prefluorescent imaging moiety; n is an integer; and the molecular weight of the carboxylated agarose polymer is 50,000-200,000, 50,000-150,000, 75,000-150,000, 80,000-140,000, 100,000-150,000, or about 120,000 amu.

In certain embodiments, the latent prefluorescent imaging moiety comprises a modified rhodamine moiety.

In certain embodiments, the modified rhodamine moiety comprises a tris(2-aminoethyl)amino moiety.

In certain embodiments, the latent prefluorescent imaging moiety has the following structure:

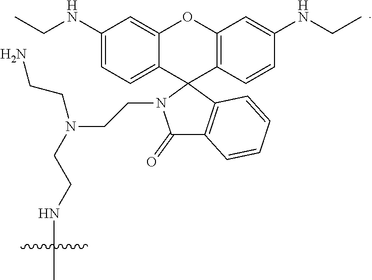 [SMILES: *NCCN(CCN)CCN1C(=O)c2ccccc2C12c1ccc(NCC)cc1Oc1cc(NCC)ccc12]

In certain embodiments, the activated prefluorescent imaging moiety that is activated upon binding to the metal ion, has the following structure:

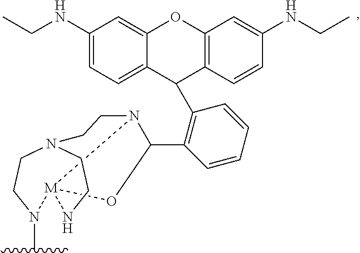

wherein M is a metal ion.

The present invention further provides a method for detecting a biomarker in an aqueous solution, the method comprising:
(1) immersing a hydrogel in the aqueous solution,
   wherein the hydrogel comprises a first biomarker capturing agent moiety;
(2) adding an imaging probe to the aqueous solution,
   wherein the imaging probe comprises a second biomarker capturing agent moiety;
(3) removing the hydrogel from the aqueous solution;
(4) dehdyrating the hydrogel; and
(5) detecting the imaging probe.

In certain embodiments, the method further comprising (6) determining the concentration of the biomarker in the aqueous solution.

In certain embodiments, the first biomarker capturing agent moiety is specific to the biomarker.

In certain embodiments, the second biomarker capturing agent moiety is specific to the biomarker.

In certain embodiments, the first biomarker capturing agent moiety is an antibody.

In certain embodiments, the second biomarker capturing agent moiety is an antibody.

In certain embodiments, the first and second biomarker capturing agent moieties are identical.

In certain embodiments, the first and second biomarker capturing agent moieties different.

In certain embodiments, the biomarker is sandwiched between the first and second biomarker capturing agents. In other embodiments, the first and second biomarker capturing agents bind to different sites on the biomarker.

In certain embodiments, the imaging probe comprises nanoparticles.

In certain embodiments, the imaging probe comprises iron oxide nanoparticles. In other embodiments, the imaging probe comprises polyacrylic-acid-coated iron oxide nanoparticles.

In certain embodiments, the iron oxide nanoparticles are detectable by X-ray fluorescence imaging.

In certain embodiments, the concentration of the biomarker in the aqueous solution and the intensity of the fluorescence of the iron oxide nanoparticles have a linear relationship.

In certain embodiments, the biomarker simultaneously binds to the hydrogel via the first capturing agent moiety and to the imaging probe via the second capturing agent moiety.

In certain embodiments, the hydrogel is size shrinkable.

In certain embodiments, the hydrogel shrinks upon dehydration.

In certain embodiments, the hydrogel comprises a modified agarose polymer.

In certain embodiments, the hydrogel comprises a carboxylated agarose polymer. In certain embodiments, the molecular weight of the carboxylated agarose polymer is 50,000-200,000, 50,000-150,000, 75,000-150,000, 80,000-140,000, 100,000-150,000, or about 120,000 amu.

In certain embodiments, each monomer of the carboxylated agarose polymer has the following structure:

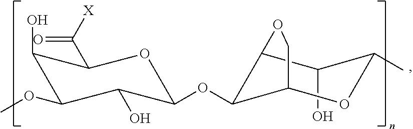

wherein X is the biomarker capturing agent moiety; n is an integer; and the molecular weight of the carboxylated agarose polymer is 50,000-200,000, 50,000-150,000, 75,000-150,000, 80,000-140,000, 100,000-150,000, or about 120,000 amu.

In certain embodiments, the hydrogel comprises vertical channels.

In certain embodiments, the biomarker is an antigen, a protein, or a nucleic acid.

In certain embodiments, the biomarker is a cancer biomarker.

### Definitions

The term “size shrinkable hydrogel” as used herein refers to a hydrogel which shrinks upon removal of water, i.e., dehydration. In general, hydrogels are highly water absorbent and increase in size when immersed in water. Size shrinkable hydrogels are able to shrink upon removal of the water.

The term “prefluorescent imaging moiety” as used herein refers to a moiety which is not capable of fluorescence in its latent state but is capable of fluorescence in its activated state. For example, in the present invention the prefluorescent imaging moiety undergoes a structural change from a spirocyclic lactam (latent state) to an open-ring amide (activated state) upon binding to lead ions.

The term “biomarker” as used herein refers broadly to a molecule found in the body that is a sign of a normal or abnormal process or of a condition or disease. Examples include, but are not limited to, proteins (e.g., an enzyme or receptor), nucleic acids, antibodies, peptides, polypeptides, and antigens. Biomarker detection may confirm the presence of a disease or condition of interest, or identify a subtype of the disease or condition. The biomarker may be present in an aqueous solution containing biological material, such as, but not limited to, blood, serum, urine, sputum, semen, saliva, tissue, fluid, ascites fluid, amniotic fluid, tissue extracts, or cell culture medium. Examples of conditions or disease, include, but are not limited to, cancers, e.g. breast cancer, ovarian cancer, lung cancer, pancreatic cancer, prostate cancer, or melanoma.

The term “biomarker-capturing agent” or “biomarker-capturing moiety” as used herein refers broadly to any agent or moiety that binds to a biomarker. Examples include, but are not limited to, antibodies and fragments thereof, antigens, aptamers, lectins, nucleic acids, and oligosaccharaides. In certain embodiments, the biomarker-capturing agent selectively binds to the biomarker. In certain embodiments, the biomarker-capturing agent or biomarker-capturing moiety is covalently bound to the polymer backbone of the hydrogel.

The term “imaging probe” as used herein refers broadly to any agent that is detectable in an imaging method. In certain embodiments, the imaging probe comprises a biomarker-capturing agent or biomarker-capturing moiety that binds to a biomarker. The detection of the imaging probe allows for a quantitative determination of the concentration of the probe in a given medium, and in turn the concentration of the biomarker. Imaging methods include, but are not limited to, fluorescence imaging and X-ray fluorescence imaging.

### Example 1: Metal Ion Detection

### Materials and Methods

Agarose powder was obtained from IBI scientific. Rhodamine 6G (99%) was obtained from Acros Organics. Lead perchlorate trihydrate [crystalline, Pb(ClO₄)₂], acetonitrile (99%), tris(2-aminoethyl)amine (97%), dichloromethane (CH₂Cl₂), methanol (>99.8%), and perfluorooctyltrichlorosilane (PFTOS, 97%) were obtained from Alfa Aesar. Tetramethyl-1-piperidinyloxy (98%, TEMPO), sodium bromide (>99.0%, NaBr), sodium hypochlorite solution (10-15%, NaClO), anhydrous sodium sulfate (NaSO₄), N-(3-dimethylaminopropyl)-N-ethylcarbodiimide (>97%, EDC), and sodium hydroxide (NaOH) were obtained from Sigma. Polydimethylsiloxane (PDMS) was obtained from Corning. Phosphate-buffered saline (PBS) was obtained from VWR.

Agarose Carboxylation. Agarose was treated with a TEMPO-NaBr—NaClO system to oxidize primary alcohol groups to CA as follows: 1 g of agarose powder was dissolved in 80 mL of water at 90° C. After adjusting the pH to 11 with 1 M aqueous NaOH, the solution was cooled down to room temperature with stirring, and 22 mL of an aqueous solution containing 0.02 g of TEMPO, 0.3 g of NaBr, and 2 mL of NaClO was added dropwise into the agarose solution, and the pH of the solution was maintained at 10-11 by adding 1 M aqueous NaOH. The oxidation reaction was completed in 1.5 h. The solution was precipitated by adding a mixture of 200 mL of isopropyl alcohol and 20 mL of acetone. The precipitate was washed twice with ethanol, and the oxidized agarose was dialyzed for 48 h and lyophilized to remove the solvent.

Grafting Turn-On Fluorescent Probes on Oxidized Agarose. A rhodamine-based turn-on prefluorescent probe was prepared (Ju, H. et al. 2011). A total of 80 mg of oxidized agarose was dissolved in 10 mL of PBS, followed by adding 40 mg of EDC to activate carboxyl groups at 80° C. for 1 h. The modified agarose was mixed with 20 mg of raw agarose to form a hydrogel, which was immersed in deionized water overnight. To graft the turn-on probe, the rinsed hydrogel was mixed with 40 μL of 2 mg/mL turn-on fluorescent probe in acetonitrile at 80° C. After reaction for 1 h, the agarose was rinsed in a mixed acetonitrile and water solution (volume ratio of 20:100) 3 times, followed by centrifugation and evaporation.

Templated Hydrogel Formation. In order to generate hydrogels with defined geometry, a bottomless well made of elastic PDMS was fabricated by using a polyacrylic well (with desired shape and dimension) as the mold. The bottomless well was firmly bonded to plasma-treated glass substrates. The fluorescent probe-immobilized hydrogel at a mass ratio of agarose and water of 1:99 was then injected into the PDMS template and taken out by separating the PDMS template and glass substrate after gelation.

Metal Ion Detection. Aqueous solutions of a variety of metal ions were prepared by dissolving corresponding salts in water with concentrations ranging from 10-1 to 10-7 M. The templated hydrogel was immersed in the solution of each metal ion at 60° C. for 30 min, followed by rinsing with acetonitrile-water (1:10). The hydrogel was dehydrated on a PFTOS-modified hydrophobic glass substrate and dried at 70° C. for 30 min.

The fluorescent images of hydrogels after catching metal ions were taken using an Olympus BX51 fluorescence microscope. The fluorescence intensity at each pixel of an image was derived by using ImageJ software. The zeta potential change of the hydrogel before and after modification was measured with a NANO ZS ZEN3600 zeta potential analyzer (Malvern Instruments). Fourier transform infrared (FTIR) spectra of hydrogels were collected with a spectrometer (Bruker Vertex 70) combined with a Hyperion 1000 microscope.

### Modified Agaroses

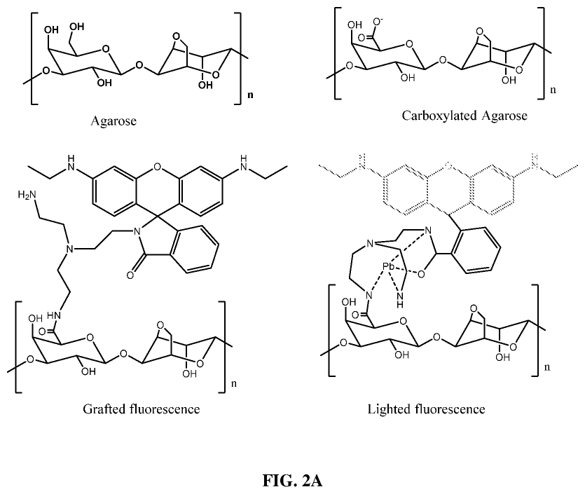

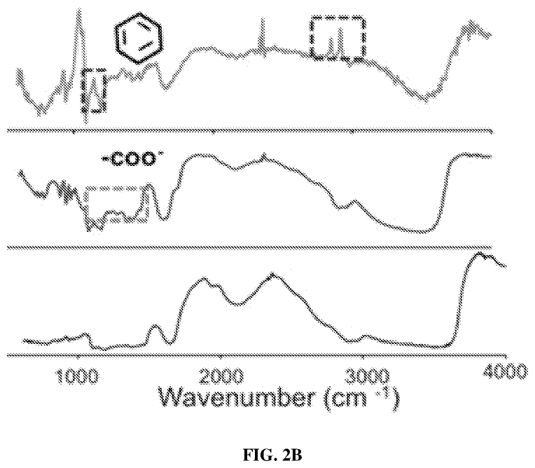

FIG. 2A shows the procedures of modifying agarose, where the primary hydroxyl groups of agarose are oxidized to carboxyl groups in CA, followed by grafting prefluorescent probes through the carboxyl-amine reaction. The prefluorescent probes do not produce fluorescence because carbon-nitrogen bonds destroy the conjugation structure in rhodamine and prevent it from fluorescence emission. Metal ions can bind to the amine group since the lone pair electrons of an amine group have a strong affinity to the outer shell of the metal ion.

Once bound to a target cation, the molecule undergoes a structural change from a spirocyclic lactam to an open-ring amide, which results in fluorescence in pink. The zeta potential of the aqueous solution of pristine agarose was nearly neutral (−2 mV), and oxidized agarose is negatively charged (−16.8 mV) due to formation of carboxyls. FTIR spectra indicated the vibrational peaks of carboxyl groups of oxidized agarose at the wavenumbers of 1400 and 1600 cm-1 (FIG. 2B). The zeta potential increased slightly (−9 mV) after immobilization of prefluorescent probes due to attachment of amines. The FTIR spectrum showed the vibrational peak of benzene on rhodamine at 1500 and 2900 cm⁻¹, which confirmed successful immobilization of prefluorescent probes.

### Metal Ion Solutions

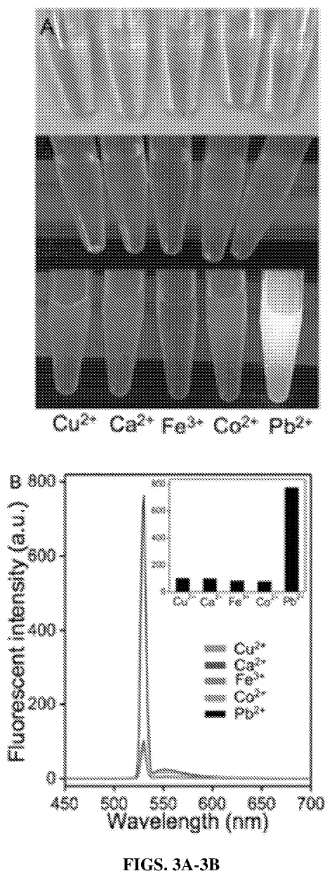

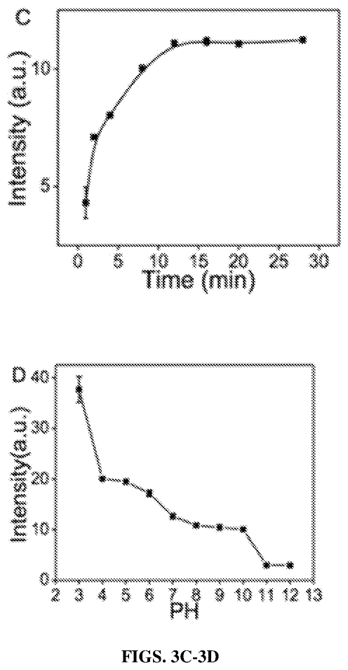

FIG. 3A shows the digital images of metal ion solutions without (upper) and with (middle) UV (365 nm) excitation, as well as with prefluorescent probes and UV light (lower). Each cuvette contained one type of 10⁻² M metal ion including iron, copper, calcium, cobalt, and lead (left to right). The fluorescent probe was found to be highly selective to lead ions. Fluorescent spectroscopy (FIG. 3B) showed that the maximum emission of the probe-lead pair is at 530 nm. The fluorescent intensity of the probe-lead pair was 10 times stronger than those of pairs formed by the probe and other ions.

FIG. 3C shows the time-dependent fluorescent intensity of the prefluorescent probe after mixing with lead ions (10⁻³M, pH of 7). The fluorescent intensity of the mixture increased with time, and reaches a plateau after 15 min.

FIG. 3D shows that the pH of the solution affects the fluorescent intensity. The fluorescent intensity was negligible when the pH is greater than 10 and when the lead ion is precipitated as lead oxide in an alkaline solvent, while the fluorescent probe was not selective to lead ions in a solution with a pH below 4. Therefore, the operational pH range of the fluorescent probe is between 4 and 10. In addition, the shrinking ability of the CA hydrogel is tested by volume measurement.

Fluorescence Emission upon Lead Binding

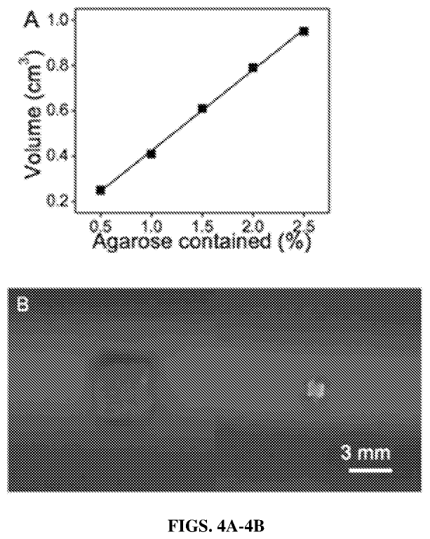

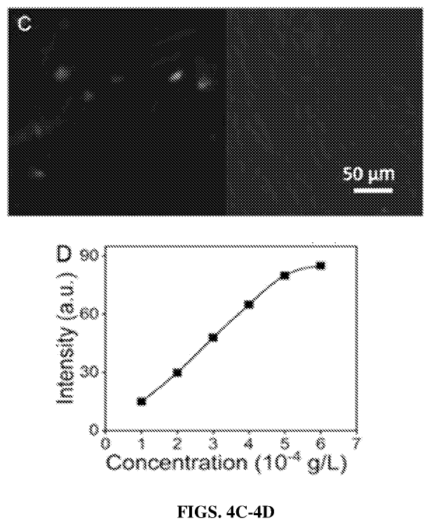

FIG. 4A shows the dry volume of CA as a function of agarose mass ratio in the original hydrogel (volume of 15 mL), where 97.5% volume reduction was achieved when 1% by weight of agarose hydrogel was used. This volume reduction could enrich molecules in dry agarose to a 40-times higher density.

FIG. 4B shows that fluorescence emission from wet agarose treated with 10⁻⁴ M lead ions was hard to see under UV light, while it was obvious in dried agarose.

FIG. 4C shows the fluorescence from the dried hydrogel observed with microscopy (right), where the fluorescence intensity was uniform across the whole gel. In contrast, the fluorescence of the hydrogel with physically adsorbed rhodamine showed uneven distribution after drying (left), suggesting that chemically immobilized fluorescence molecules undergo the same volume change as the hydrogel itself and do not aggregate during drying.

FIG. 4D shows that a linear relation exists between fluorescence intensity and the concentration of fluorescence probes in the hydrogel, where an excess amount of lead ions is added to ensure all prefluorescent probes are lighted. Therefore, the fluorescence intensity should be exponential to the level of lead ions.

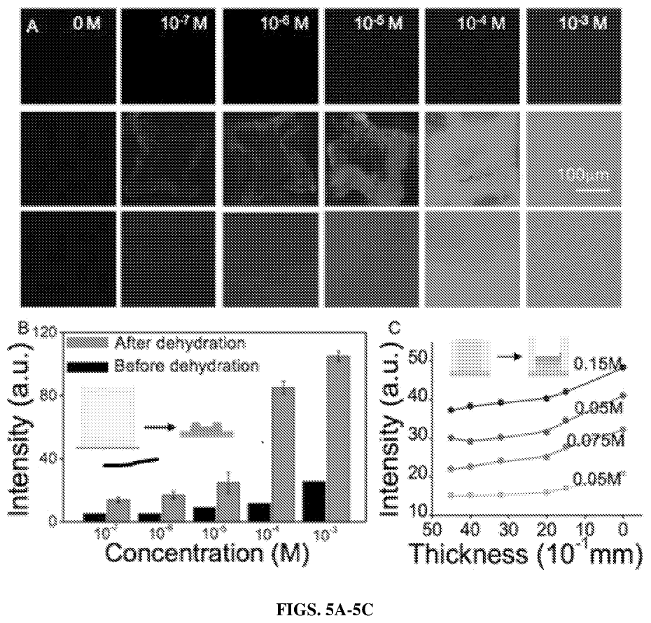

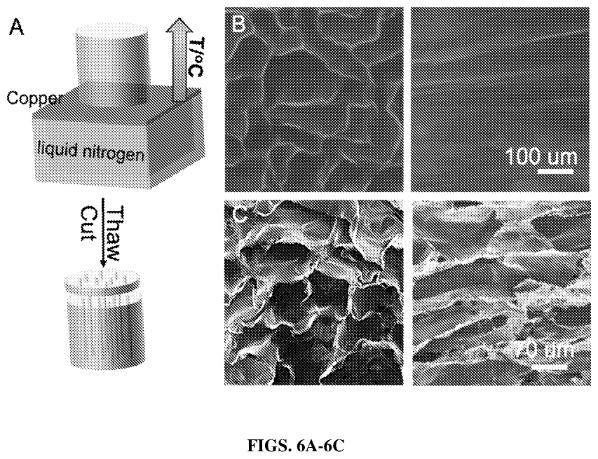

FIG. 5A shows the fluorescence images of lighted hydrogels with different loads of lead ions before (upper) and after drying (middle) taken at the same UV excitation and exposure time (100 ms), confirming that the hydrogel dehydration enhanced fluorescence. Meanwhile, the cubic hydrogel dries to achieve a rectangular shape because water evaporation occurs preferentially at the sides of the rectangle (due to large contact areas with air), which produced a mass transfer of the wet hydrogel from the center to the edge (FIG. 5B). The detection sensitivity to lead ions is enhanced to 10⁻⁷ M by comparing the fluorescence contrasts of the edge of the dry hydrogel and background. The fluorescence intensity at the edge of a dried rectangular-shaped gel was derived by ImageJ. The intensity of fluorescence depends on the amount of the fluorescence probe and geometry of the dried hydrogel.

FIG. 5B shows an exponential dependence of the fluorescence intensity on the lead ion level (from 10⁻⁷ to 10⁻³ M), where red (right) and black (left) bars are fluorescent intensities of hydrogels before and after dehydration, respectively. The enhancement ranges from 3 times at low concentrations to more than 10 times at high concentrations, compared to those of wet hydrogels.

FIG. 5C shows the fluorescence intensity of the hydrogel in a vertical direction as a function of thickness during the dehydration process, where the hydrogel was confined inside a Petri dish to allow evaporation only through the top surface. The fluorescent intensity increased by 20% during dehydration, which is much smaller than the lateral direction. The relatively small increase in fluorescence is because the number of fluorescence probes in the vertical direction does not increase much after dehydration. Therefore, the fluorescence intensity should be exponential to the level of lead ions. The US Environmental Protection Agency regulates that the lead ions in drinking water should be below 10⁻⁷ M; thus, this method allows visual examination against lead ions in drinking water.

### Example 2: Biomarker Detection

### Materials and Methods

Disuccinimidyl suberate (DSS), biotin-PEG₂-amine and avidin were from Thermofisher. Phosphate buffered saline (PBS) was from VWR and tween 20 was from Acros. Bovine serum albumin (BSA), dimethyl sulfoxide (DMSO, 99%) and perfluorooctyltrichlorosilane (PFTOS, 97%) were from AlfaAesar. The following chemicals were from Sigma-Aldrich: tetramethyl-1-piperidinyloxy (TEMPO, 98%), N-(3-dimethylaminopropyl)-N′-ethylcarbodiimide (EDC, 97%), N-Hydroxysuccinimide (NHS), (3-aminopropyl) triethoxysilane (APTES), sodium hydroxide (NaOH), sodium bromide (NaBr, 99.0%), sodium hypochlorite solution (NaClO, 10-15%), and iron oxide nanoparticles (25 nm diameter, 5 mg/mL). Agarose powder was from IBI Scientific.

Chemical Modification of Agarose—Agarose was treated with TEMPO-NaBr—NaClO to oxidize the primary alcohol groups to carboxylated agarose (CA) as follows. 1 g of agarose powder was dissolved in 80 mL of water at 90° C. After adjusting pH to 11 with 1M of aqueous NaOH, the solution was cooled to room temperature while stirring. 22 mL of an aqueous solution containing 0.02 g TEMPO, 0.3 g NaBr, and 2 mL NaClO was then added dropwise into the agarose solution, and the pH of the solution was maintained at 10-11 by adding 1 M NaOH solution. The oxidation reaction was completed in 1.5 hours. The solution was precipitated by adding a mixture of 200 mL of isopropyl alcohol and 20 mL of acetone. The precipitate (i.e., oxidized agarose) was washed twice with ethanol, dialyzed for 48 hours and lyophilized to remove solvent.

Carboxylation of hydrogel scaffold—Freeze-thaw method was used to make hydrogel scaffolds with vertical channels as follows. 0.05 g of carboxylated agarose (CA) and 0.05 g of agarose were dissolved in 10 mL of water at 100° C., followed by gelation at room temperature. The hydrogel was made into a cylinder of 1.5 cm in diameter, cut as 2.5 cm in length and placed on a copper block, which sits at the top of a foam box. The freezing process started when liquid nitrogen was filled in the box, creating a uniaxial thermal gradient. The samples were allowed to freeze for 10 min and removed from the copper block to thaw at room temperature, followed by cutting to thin slabs of 1.5 mm thick.

Biotin-conjugated iron oxide nanoparticles—0.2 mL of aqueous solution of iron oxide nanoparticles was mixed with 5 mL of alcoholic solution of APTES (0.2% by weight) for 2 hours under stirring. After removing excess chemicals by centrifuge, nanoparticles were suspended in 2 mL of DMSO containing 4 mg of DSS as the crosslinker for 1 hour at 37° C. The solution was added into a 2 mL aqueous solution of biotin-PEG2-amine (containing 10 mg of biotin-PEG₂-amine) for 2 hours. Biotin modified nanoparticles were then rinsed water and centrifuged for three times. Fluorescent images of nanoparticles were taken with an Olympus BX51 fluorescence microscope. Scanning electron microscopy (SEM) images were taken by high-resolution field emission scanning electron microscope Hitachi S-4800. Attenuated total reflectance Fourier transform infrared (ATR-FTIR) spectra were collected with a Thermo Scientific Nicolet iS10 FTIR spectrometer.

Protein detection—The schematic detection processing is shown in Figure. Avidin was dissolved in the sterile water at concentrations ranging from 0.2 [symbol] g/mL to 2 mg/mL. The carboxylated agarose was immersed in the avidin solution for an hour at 37° C. and then rinsed by using 0.1% tween 20 and water three times. In order to block carboxyl groups on hydrogel, the carboxylated agarose was then immersed in an aqueous solution of BSA (0.5% by weight) for 1 hour at 37° C., followed by rinsing with tween 20 solution and water. In order to image the avidin modified hydrogel, iron oxide nanoparticles with biotin labels were added into the scaffold for 30 min. The unbounded nanoparticles were removed from tween 20 solutions using a magnet. The scaffold was then placed on a PFTOS modified glass slide and dehydrated in an oven at 50° C.

X-ray fluorescence detection—A Mini-X X-ray tube (Amptek, Bedford Mass.) operating at 40 kV and 15 A was used to generate primary X-rays. The tube was fitted with a brass collimator to reduce the beam size to 1 mm in diameter. An X-ray spectrometer (Amptek X-123) with Si—PIN photodiode was used to collect X-ray fluorescence (XRF) signals in reflection modes, where the tube and the detector were fixed on a mounting plate with an angle of 45° between them. The tube-sample distance and sample detector distance are kept at 3 and 2 cm respectively, and the setup was enclosed in a lead containing acrylic chamber.

### Analysis of Hydrogel

The hydrogel was made into a cylinder of 1.5 cm in diameter, cut as 2.5 cm in length and placed on a copper block, which sits at the top of a foam box (FIG. 2A). The carboxylated agarose hydrogel made with freeze-thaw method was colored by Rhodamine 6G and examined by a fluorescent microscope. FIG. 2B shows that the hydrogel scaffold has uniaxial channels along the length with an average width of 150 μm. The scaffold is then lyophilized and imaged by SEM, which shows the channel width is about 120 μm. A 20% reduction in channel width due to volume shrinkage during lyophilization can be derived from FIG. 2C.

The diffusivity of a molecule in the hydrogel was derived by observing the distance of the front of a fluorescently labeled dye with time while partially immersing the hydrogel in the solution of fluorescent molecules.

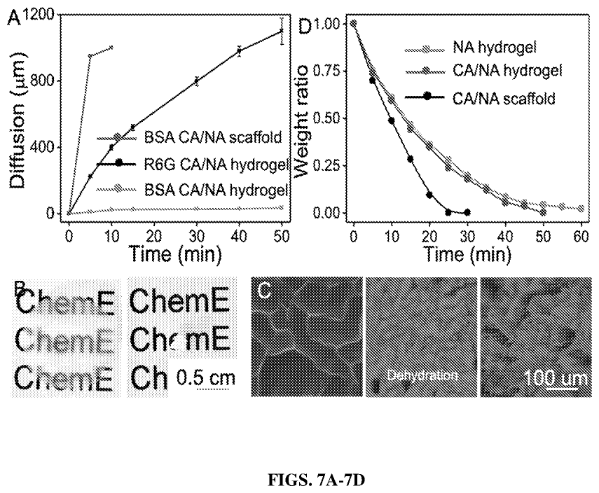

FIG. 7A shows the diffusivities of rhodamine 6G itself and rhodamine 6G labeled BSA, where (1) rhodamine 6G shows higher diffusivity on hydrogel with random pores (porous hydrogel) and hydrogel with channel structures (construct) than BSA due to smaller size of rhodamine 6G compared to BSA; (2) both rhodamine 6G and BSA show higher diffusivities in hydrogel with channel structures than hydrogel with random pores.

FIG. 7B shows the optical images of hydrogel scaffolds before and after dehydration, where the scaffold changes from transparent to opaque, and its volume decreases to 1/80 times. The fluorescence images (FIG. 7C) of the cross section areas of a dehydrated scaffold are taken at three time points during dehydration, where the cross section area becomes smaller as dehydration progresses. FIG. 3D shows the relationship between water weight ratio and time on a hydrogel and a scaffold that have the same shape and size, where the scaffold dehydrates in the least time, meaning the fastest dehydration.

### Iron Oxide Particles

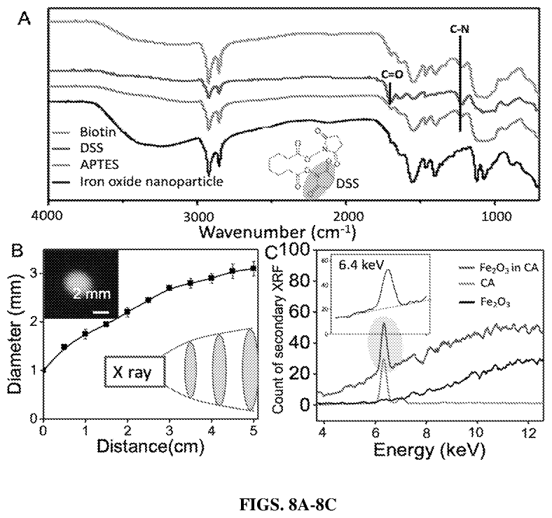

The iron oxide nanoparticles were modified to have biotin at the outmost. FIG. 8A shows ATR-FTIR spectra collected after modification using pristine nanoparticles as references. APTES modified nanoparticles show characteristic C—N stretching vibration of amine on APTES at 1275 cm⁻¹ (green). DSS covalently linked on APTES presents the vibrational peak of C═O from its free NHS ester at 1725 cm⁻¹ (blue). ATR-FTIR spectrum also shows the attenuation of C═O vibrational peak at 1725 cm⁻¹ after covalent binding of NHS ester of DSS to amine of biotin-PEG-amine (red). The incident X-ray beam should match the size of the sample. The size of a cone shaped X-ray beam is controlled by changing the distance from the X-ray tube and the sample.

FIG. 8B (insert) shows an optical image of a round fluorescent spot (diameter of 2.5 mm) produced on an X-ray sensitive film placed at 2.5 cm from the X-ray tube. FIG. 4B shows the relation between spot size of X-ray beam and distance, where the spot size decreases as the distance decreases.

FIG. 8C shows the XRF spectrum of iron oxide (red), dehydrated hydrogel (black) and iron oxide bonded dehydrated hydrogel (blue), collected at an accumulation time of 100 s at a distance of 2.5 cm between the X-ray tube and the sample. Iron oxide sample shows the Ka peak of iron at 6.40 keV and a small Kb peak at 7.06 keV with the area ratio 6.6:1 in the spectrum. Dehydrated hydrogel shows the noise peaks of the samples, which could be the scattering of the incoming X-rays on the rough dehydrated hydrogel surface. The noise peaks of the dehydrated hydrogels immerse the Kb peak of the iron in the spectrum for iron oxide bonded dehydrated hydrogels. The inserted image shows the iron peak and its baseline. The XRF signal of oxygen is at the low energy side of the spectrum, which cannot be distinguished.

### Detection of Protein Antibodies

The detection of protein antibodies was performed on channel hydrogels as specified in the materials section. The area of the XRF peak of iron is used to derive the amount of iron oxide nanoparticles attached to hydrogel gel.

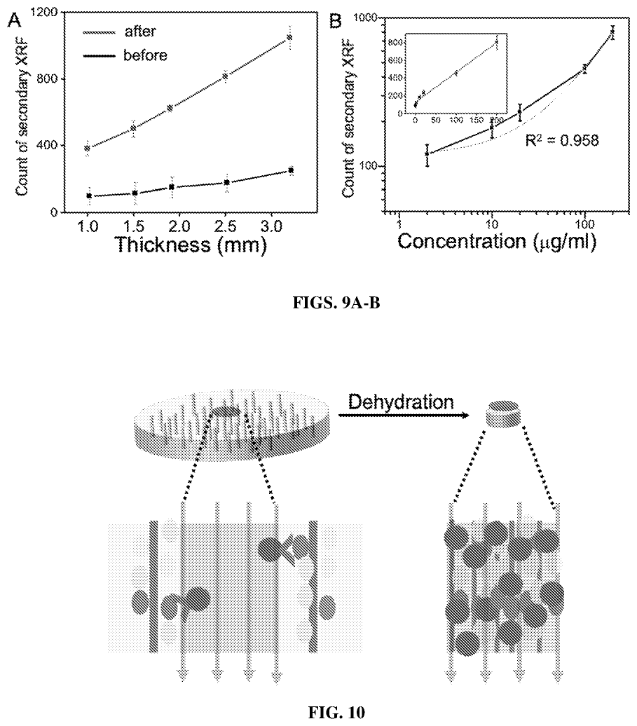

FIG. 9A shows the relationship between the peak area of iron oxide nanoparticles before (black) and after (blue) the dehydration of hydrogel as a function of hydrogel thickness. The peak areas increase as the thicknesses of hydrogels increase in both hydrogels and dehydrated gels. A six-fold increase in the peak area is found in the fully dehydrated sample compared to the hydrogels, which confirms the sensitivity enhancement of XRF detection after hydrogel dehydration. Other than sensitivity, the hydrogel thickness should be considered, because thicker hydrogel consumes more analytes and nanoparticles.

In the next experiment to detect avidin, the thickness of hydrogel is set at 1.5 mm and a magnet is used to remove unbounded nanoparticles prior to hydrogel dehydration. FIG. 9B shows the peak areas of hydrogels after detecting avidin with concentration ranging from 2 mg/mL to 2 μg/mL. A linear relation exists, which is shown as the fitted red lines. The inserted image shows the data in a linear axis, which indicates the limitation of detection is 2 μg/mL for avidin compared to the background (no avidin). The ranging from 2 mg/mL to 2 μg/mL could be regarded as linear range of the detection due to the scarcity of the avidin and nanoparticles.

### DISCUSSION

The volume reduction of hydrogels upon dehydration was used to enhance the sensitivity of turn-on fluorescence detection of lead ions in water. The rhodamine-derived fluorescent probes grafted on CA hydrogel showed excellent selectivity to lead ions and superior turn-on fluorescence upon binding lead ions. The dehydration of hydrogel lead to a volume reduction of more than 40 times and can effectively increase fluorescence emission to 10 times higher after capturing lead ions at a concentration of 10⁻⁷ M. The enhancement is at such a degree that the turn-on fluorescence upon lead ion binding was easily seen with naked eyes. Given its low cost, straightforwardness, easy operation, and high sensitivity, this volume-changing hydrogel can be used to detect lead ions in drinking water.

The volume reduction of hydrogels upon dehydration was also employed to enhance the sensitivity of X-ray fluorescence detection of biomarkers. A method is disclosed to enhance the sensitivity of nanoparticle-based protein detection using size-shrinkable hydrogels, in which X-ray fluorescence nanoparticles are captured on a hydrogel scaffold that has aligned channel structures (FIG. 10). The channels allow rapid diffusion of water, nanoparticles, and protein targets. The surfaces of the hydrogel is modified with specific antibodies as capturing agents; the surfaces of nanoparticles are modified with the secondary antibodies. In the presence of target antigens, the nanoparticles are immobilized on the hydrogel via antigen-antibody interactions. After rinsing away the unbound nanoparticles, the hydrogel is dehydrated to reduce its volume, followed by detecting the intensity of X-ray fluorescence from nanoparticles. The nanoparticles used herein (iron oxide nanoparticles) were chosen for their relatively high X-ray fluorescence yield and readiness of being removed with a magnet if they are not bound on hydrogel (Manohar, N. et al. 2016; Wu, D. et al. 2013; Hossain, M. et al. 2010; Landmark K. J. et al. 2008).

The area density or concentration of nanoparticles is increased by use of size-reducible hydrogels, whose volume can be reduced tens or even hundreds of times upon dehydration (Wu, Z. L., et al. 2013; Kudo, K. et al. 2014). This approach was used to increase the sensitivity of metal ion detection by using organic fluorophore; however, extending this technique to nanoparticle probes for biomolecule detection remains challenging because the diffusion resistances of biomolecules and nanoparticles into porous constructs are significant. A temperature gradient controlled ice crystallization was developed to produce porous scaffolds with vertically aligned channels that extend the full length of hydrogel thin film (Stokols, S. et al. 2006; Hua, M. et al. 2021). Carboxylated agarose hydrogel scaffolds, composed of uniaxial channels extending through their entire length, allow high diffusivity of biomolecules and fast dehydration.

### REFERENCES CITED

Appleyard, D. C., et al., Bar-coded hydrogel microparticles for protein detection: synthesis, assay and scanning. Nature Protocols, 2011. 6 (11): p. 1761-1774.
Axpe, E. et al., A Multiscale Model for Solute Diffusion in Hydrogels. Macromolecules, 2019. 52 (18): p. 6889-6897.
Burdick, J. A. and G. D. Prestwich, Hyaluronic Acid Hydrogels for Biomedical Applications. Advanced Materials, 2011. 23(12): p. H41-H56.
Burnham, M. R.; Turner, J. N.; Szarowski, D.; Martin, D. L. Biological functionalization and surface micropatterning of polyacrylamide hydrogels. Biomaterials 2006, 27, 5883-5891.
Chen, J.; Teo, K. C. Determination of cadmium, copper, lead and zinc in water samples by flame atomic absorption spectrometry after cloud point extraction. Anal. Chim. Acta 2001, 450, 215-222.
Chen, F.; Tillberg, P. W.; Boyden, E. S. Expansion microscopy. Science 2015, 347, 543.
Czaplyski, W. L.; Purnell, G. E.; Roberts, C. A.; Allred, R. M.; Harbron, E. J. Substituent effects on the turn-on kinetics of rhodamine-based fluorescent pH probes. Org. Biomol. Chem. 2014, 12, 526-533.
Dadsetan, M., et al., Effect of hydrogel porosity on marrow stromal cell phenotypic expression. Biomaterials, 2008. 29(14): p. 2193-2202.
Dehghani, F. and N. Annabi, Engineering porous scaffolds using gas-based techniques. Current Opinion in Biotechnology, 2011. 22(5): p. 661-666.
Deo, S.; Godwin, H. A. A selective, ratiometric fluorescent sensor for Pb2+. J. Am. Chem. Soc. 2000, 122, 174-175.
Dhenadhayalan, N.; Lee, H.-L.; Yadav, K.; Lin, K.-C.; Lin, Y.-T.; Chang, A. H. H. Silicon quantum dot-based fluorescence turn-on metal ion sensors in live cells. ACS Appl. Mater. Interfaces 2016, 8, 23953-23962.
De France, K. J., F. Xu, and T. Hoare, Structured Macroporous Hydrogels: Progress, Challenges, and Opportunities. Advanced Healthcare Materials, 2018. 7(1): p. 1700927.
Forget, A.; Pique, R.-A.; Ahmadi, V.; Lüdeke, S.; Shastri, V. P. Mechanically tailored agarose hydrogels through molecular alloying with β-sheet polysaccharides. Macromol. Rapid Commun. 2015, 36, 196-203.
Futscher, M. H.; Philipp, M.; Müller-Buschbaum, P.; Schulte, A. The Role of Backbone Hydration of Poly(N-isopropyl acrylamide) Across the Volume Phase Transition Compared to its Monomer. Sci. Rep. 2017, 7, 17012.
Garner, B. W.; Cai, T.; Ghosh, S.; Hu, Z.; Neogi, A. Refractive Index Change Due to Volume-Phase Transition in Polyacrylamide Gel Nanospheres for Optoelectronics and Bio-photonics. Appl. Phys. Express 2009, 2, 057001.
Gong, J.; Schuurmans, C. C. L.; Genderen, A. M. v.; Cao, X.; Li, W.; Cheng, F.; He, J. J.; López, A.; Huerta, V.; Manríquez, J.; Li, R.; Li, H.; Delavaux, C.; Sebastian, S.; Capendale, P. E.; Wang, H.; Xie, J.; Yu, M.; Masereeuw, R.; Vermonden, T.; Zhang, Y. S. Complexation induced resolution enhancement of 3D-printed hydrogel constructs. Nat. Commun. 2020, 11, 1267.
Goryacheva, I. Y.; Shtykov, S. N.; Loginov, A. S.; Panteleeva, I. V. Preconcentration and fluorimetric determination of polycyclic aromatic hydrocarbons based on the acid-induced cloud-point extraction with sodium dodecylsulfate. Anal. Bioanal. Chem. 2005, 382, 1413-1418.
Greda, K.; Swiderski, K.; Jamroz, P.; Pohl, P. Flowing liquid anode atmospheric pressure glow discharge as an excitation source for optical emission spectrometry with the improved detectability of Ag, Cd, Hg, Pb, Tl, and Zn. Anal. Chem. 2016, 88, 8812-8820.
Guenther, M.; Kuckling, D.; Corten, C.; Gerlach, G.; Sorber, J.; Suchaneck, G.; Arndt, K. Chemical sensors based on multiresponsive block copolymer hydrogels. Sens. Actuators, B 2007, 126, 97-106.
Hossain, M., C. Wang, and M. Su, Multiplexed biomarker detection using x-ray fluorescence of composition-encoded nanoparticles. Applied Physics Letters, 2010. 97(26): p. 263704.
Hua, M., et al., Strong tough hydrogels via the synergy of freeze-casting and salting out. Nature, 2021. 590 (7847): p. 594-599.
Hu, J.-X.; Ran, J.-B.; Chen, S.; Jiang, P.; Shen, X.-Y.; Tong, H. Carboxylated agarose (CA)-silk fibroin (SF) dual confluent matrices containing oriented hydroxyapatite (HA) crystals: biomimetic organic/inorganic composites for tibia repair. Biomacromolecules 2016, 17, 2437-2447.
Ingavle, G. C.; Gehrke, S. H.; Detamore, M. S. The bioactivity of agarose-PEGDA interpenetrating network hydrogels with covalently immobilized RGD peptides and physically entrapped aggrecan. Biomaterials 2014, 35, 3558-3570.
Javed, H.; Islam, A.; Chauhan, A.; Kumar, S.; Kumar, S. Efficacy of engineered GO Amberlite XAD-16 picolylamine sorbent for the trace determination of Pb (II) and Cu (II) in fishes by solid phase extraction column coupled with inductively coupled plasma optical emission spectrometry. Sci. Rep. 2018, 8, 17560.
Ju, H.; Lee, M. H.; Kim, J.; Kim, J. S.; Kim, J. Rhodamine-based chemosensing monolayers on glass as a facile fluorescent “turn-on” sensing film for selective detection of Pb²+. Talanta 2011, 83, 1359-1363.
Kim, Y.; Jang, G.; Lee, T. S. New Fluorescent Metal-Ion Detection Using a Paper-Based Sensor Strip Containing Tethered Rhodamine Carbon Nanodots. ACS Appl. Mater. Interfaces 2015, 7, 15649-15657.
Kudo, K., et al., Structural changes of water in poly(vinyl alcohol) hydrogel during dehydration. The Journal of Chemical Physics, 2014. 140(4): p. 044909.
Kumar, A., et al., Cryogels: Freezing unveiled by thawing. Materials Today, 2010. 13(11): p. 42-44.
Landmark, K. J., et al., Synthesis, Characterization, and in Vitro Testing of Superparamagnetic Iron Oxide Nanoparticles Targeted Using Folic Acid-Conjugated Dendrimers. ACS Nano, 2008. 2(4): p. 773-783.
Lee, H. J., A. W. Wark, and R. M. Corn, Microarray methods for protein biomarker detection. Analyst, 2008. 133(8): p. 975-983.
Lenzi, E., D. Jimenez de Aberasturi, and L. M. Liz-Marzán, Surface-Enhanced Raman Scattering Tags for Three-Dimensional Bioimaging and Biomarker Detection. ACS Sensors, 2019. 4(5): p. 1126-1137.
Li, Z.-h.; Liu, R.; Tan, Z.-l.; He, L.; Lu, Z.-l.; Gong, B. Aromatization of 9,10-dihydroacridine derivatives: discovering a highly selective and rapid-responding fluorescent probe for peroxynitrite. ACS Sens. 2017, 2, 501-505.
Lu, H.; Qi, S.; Mack, J.; Li, Z.; Lei, J.; Kobayashi, N.; Shen, Z. Facile Hg²⁺ detection in water using fluorescent self-assembled monolayers of a rhodamine-based turn-on chemodosimeter formed via a “click” reaction. J. Mater. Chem. 2011, 21, 10878-10882.
Madihally, S. V. and H. W. T. Matthew, Porous chitosan scaffolds for tissue engineering. Biomaterials, 1999. 20(12): p. 1133-1142.
Madrid, Y.; Camara, C. Lead hydride generation atomic absorption spectrometry: an alternative to electrothermal atomic absorption spectrometry. A review. Analyst 1994, 119, 1647-1658.
Mandil, A.; Idrissi, L.; Amine, A. Stripping voltammetric determination of mercury(II) and lead(II) using screen-printed electrodes modified with gold films, and metal ion preconcentration with thiol-modified magnetic particles. Microchim. Acta 2010, 170, 299-305.
Manohar, N., et al., Quantitative imaging of gold nanoparticle distribution in a tumor-bearing mouse using benchtop x-ray fluorescence computed tomography. Scientific Reports, 2016. 6(1): p. 22079.
Meiring, J. E., et al., Hydrogel Biosensor Array Platform Indexed by Shape. Chemistry of Materials, 2004. 16(26): p. 5574-5580.
Pierre, G.; Punta, C.; Delattre, C.; Melone, L.; Dubessay, P.; Fiorati, A.; Pastori, N.; Galante, Y. M.; Michaud, P. TEMPO-mediated oxidation of polysaccharides: An ongoing story. Carboydr. Polym. 2017, 165, 71-85.
Rapsomanikis, S.; Donard, 0. F. X.; Weber, J. H. Speciation of lead and methyllead ions in water by chromatography/atomic absorption spectrometry after ethylation with sodium tetraethylborate. Anal. Chem. 1986, 58, 35-38.
Sanders, T.; Liu, Y.; Buchner, V.; Tchounwou, P. B. Neurotoxic effects and biomarkers of lead exposure: a review. Rev. Environ. Health 2009, 24, 15-45.
Sandrin, D., et al., Diffusion of macromolecules in a polymer hydrogel: from microscopic to macroscopic scales. Physical Chemistry Chemical Physics, 2016. 18(18): p. 12860-12876.
Savina, I. N., et al., A simple method for the production of large volume 3D macroporous hydrogels for advanced biotechnological, medical and environmental applications. Scientific Reports, 2016. 6(1): p. 21154.
Shi, D.; Yan, F.; Zhou, X.; Zheng, T.; Shi, Y.; Fu, W.; Chen, L. Preconcentration and fluorometric detection of mercury ions using magnetic core-shell chitosan microspheres modified with a rhodamine spirolactam. Microchim. Acta 2016, 183, 319-327.
Stokols, S. and M. H. Tuszynski, Freeze-dried agarose scaffolds with uniaxial channels stimulate and guide linear axonal growth following spinal cord injury. Biomaterials, 2006. 27(3): p. 443-451.
Su, Y.; Chu, B.; Gao, Y.; Wu, C.; Zhang, L.; Chen, P.; Wang, X.; Tang, S. Modification of agarose with carboxylation and grafting dopamine for promotion of its cell-adhesiveness. Carbohydr. Polym. 2013, 92, 2245-51.
Wan, J.; Zhang, K.; Li, C.; Li, Y.; Niu, S. A novel fluorescent chemosensor based on a rhodamine 6G derivative for the detection of Pb²⁺ ion. Sens. Actuators, B 2017, 246, 696-702.
Wani, A. L.; Ara, A.; Usmani, J. A. Lead toxicity: a review. Interdiscip. Toxicol. 2015, 8, 55-64.
Wu, D., et al., A method of measuring gold nanoparticle concentrations by x-ray fluorescence for biomedical applications. Medical Physics, 2013. 40(5): p. 051901.
Wu, J.; Boyle, E. A. Low blank preconcentration technique for the determination of lead, copper, and cadmium in small-volume seawater samples by isotope dilution ICPMS. Anal. Chem. 1997, 69, 2464-2470.
Wu, Z. L.; Moshe, M.; Greener, J.; Therien-Aubin, H.; Nie, Z.; Sharon, E.; Kumacheva, E. Three-dimensional shape transformations of hydrogel sheets induced by small-scale modulation of internal stresses. Nat. Commun. 2013, 4, 1586.
Xue, F.; Meng, Z.; Wang, F.; Wang, Q.; Xue, M.; Xu, Z. A 2-D photonic crystal hydrogel for selective sensing of glucose. J. Mater. Chem. A 2014, 2, 9559-9565.
Yin, J.; Ma, X.; Wei, G.; Wei, D.; Du, Y. A highly selective and sensitive sugar-rhodamine “turn-on” fluorescent sensor for divalent copper ion detection in acetonitrile. Sens. Actuators, B 2013, 177, 213-217.
Zubtsov, D. A., et al., Comparison of surface and hydrogel-based protein microchips. Analytical Biochemistry, 2007. 368(2): p. 205-213.

### INCORPORATION BY REFERENCE

All of the U.S. patents and U.S. and PCT published patent applications cited herein are hereby incorporated by reference.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

1. A method for detecting a metal ion in an aqueous solution, the method comprising:
(1) immersing a hydrogel in the aqueous solution,
wherein the hydrogel comprises a latent imaging moiety that is activated upon binding the metal ion;
(2) removing the hydrogel from the aqueous solution;
(3) dehydrating the hydrogel; and
(4) detecting the activated imaging moiety.

2. The method of claim 1, further comprising (5) determining the concentration of the metal ion in the aqueous solution.

3. The method of claim 1, wherein the metal ion is a Pb²⁺ ion.

4. (canceled)

5. The method of claim 1, wherein the hydrogel shrinks upon dehydration.

6. A method for detecting a metal ion in an aqueous solution, the method comprising:
(1) immersing a hydrogel in the aqueous solution,
wherein the hydrogel comprises a latent prefluorescent imaging moiety that is activated upon binding the metal ion;
(2) removing the hydrogel from the aqueous solution;
(3) dehydrating the hydrogel; and
(4) detecting the fluorescence emitted by the activated prefluorescent imaging moiety.

7. The method of claim 6, further comprising (3a) exposing the dehydrated hydrogel to UV light.

8. The method of claim 6, further comprising (5) determining the concentration of the metal ion in the aqueous solution.

9. (canceled)

10. The method of claim 6, wherein the metal ion is a Pb²⁺ ion.

11. (canceled)

12. The method of claim 6, wherein the hydrogel shrinks upon dehydration.

13. The method of claim 6, wherein in step (1), the hydrogel is immersed in the aqueous solution for about 20 to about 60 min.

14. (canceled)

15. The method of claim 6, wherein in step (1), the aqueous solution containing the immersed hydrogel is heated to about 50 to 80° C.

16. (canceled)

17. (canceled)

18. The method of claim 6, wherein in step (2), the hydrogel is dehydrated at about 60 to about 90° C.

19. (canceled)

20. The method of claim 18, wherein the hydrogel is dehydrated for about 20 to about 60 min.

21. (canceled)

22. (canceled)

23. (canceled)

24. (canceled)

25. The method of claim 6, wherein the intensity of the fluorescence emitted by the activated prefluorescent imaging moiety in the dehydrated hydrogel is about 10 times greater relative to the wet hydrogel.

26. The method of claim 1, wherein the hydrogel comprises a modified agarose polymer which comprises a carboxylated agarose polymer.

27. (canceled)

28. The method of claim 26, wherein each monomer of the carboxylated agarose polymer has the following structure: 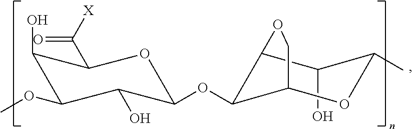
wherein X is the prefluorescent imaging moiety; n is an integer; and the molecular weight of the carboxylated agarose polymer is 50,000-200,000, 50,000-150,000, 75,000-150,000, 80,000-140,000, 100,000-150,000, or about 120,000 amu.

29. The method of claim 28, wherein the latent prefluorescent imaging moiety comprises a modified rhodamine moiety which comprises a tris(2-aminoethyl)amino moiety.

30. (canceled)

31. The method of claim 29, wherein the latent prefluorescent imaging moiety has the following structure:

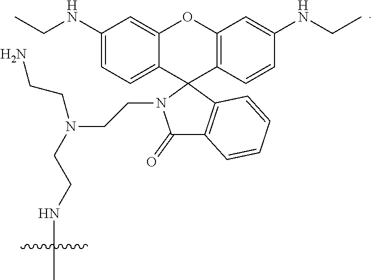 [SMILES: *NCCN(CCN)CCN1C(=O)c2ccccc2C12c1ccc(NCC)cc1Oc1cc(NCC)ccc12]

32. The method of claim 6, wherein the activated prefluorescent imaging moiety that is activated upon binding to the metal ion, has the following structure: 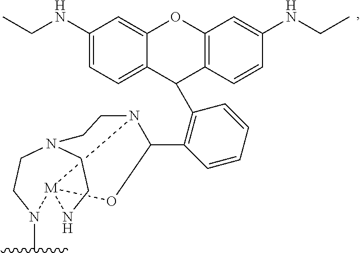
wherein M is a metal ion.

33.-44. (canceled)

45. A method for detecting a biomarker in an aqueous solution, the method comprising:
(1) immersing a hydrogel in the aqueous solution,
wherein the hydrogel comprises a first biomarker capturing agent moiety;
(2) adding an imaging probe to the aqueous solution,
wherein the imaging probe comprises a second biomarker capturing agent moiety;
(3) removing the hydrogel from the aqueous solution;
(4) dehdyrating the hydrogel; and
(5) detecting the imaging probe.

46.-65. (canceled)
